(19) United States
(12) Patent Application Publication
(10) Pub. No.: US 2007/0168041 A1
(43) Pub. Date: Jul. 19, 2007

(54) Method and instruments for intervertebral disc augmentation through a pedicular approach

(76) Inventor: Sudhakar Kadiyala, Newton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/333,589

(22) Filed: Jan. 17, 2006

(51) Int. Cl. A61F 2/44

(52) U.S. Cl. 623/17.16; 623/908

(57) ABSTRACT

A method of replacing a nucleus pulposus in an intervertebral disc by filling the disc with a flowable augmentation material through a throughbore in a pedicle.

### BACKGROUND OF THE INVENTION

As a therapy for degenerative disc disease (“DDD”), some investigators have proposed removing at least a portion of the degraded nucleus pulposus and replacing it with a nucleus augmentation material.

Current techniques for disc augmentation rely upon delivery of the disc augmentation material through the annulus fibrosus portion of the disc. There are two principle approaches in delivering disc augmentation material. In one approach, large structural elements, such as expandable hydrogel pillows, are introduced through relatively large holes created in the annulus fibrosus. One problem with this approach is that access to the nucleus pulposus requires breach of the annulus fibrosus, an avascular cartilaginous structural that heals slowly, if at all. The resulting hole in the annulus fibrosus may serve as a conduit for extravasation and leakage of the disc augmentation material. Accordingly, the hole in the annulus fiborsus must be repaired, typically by suturing. However, such repair is often imperfect and there remains a risk of expulsion of the nucleus augmentation materials through the sutured hole.

Another augmentation approach uses an injectable material that can be delivered across the annulus fibrosus through a needle. Despite the smaller size of the insult to the annulus fibrosus, leakage through the hole in the annulus fibrosus remains a concern. In this latter approach, materials that change phase (e.g., solidify) after their injection into the disc are being proposed as a means to prevent leakage. However, this drastically limits the types of materials that can be used for disc augmentation.

U.S. Pat. No. 6,685,695 (“Ferree”) discloses providing a stent in the intervertebral disc that allows nutrition to flow from the adjacent vertebral bodies into the disc. Ferree does not disclose the entry route of the stent into the disc.

US Published Patent Application No. 2005/0125066 (“McAfee”) discloses systems for nucleus pulposus replacement by constructing channels through the pedicles. In McAfee's third embodiment, the pedicles of the same vertebra are used as access into the adjacent disc space. McAfee teaches that this allows preservation of the entire periphery of the annulus fibrosus if the nucleus replacement is collapsible and can be inserted through the pedicle. In each embodiment, McAfee requires the use of a suture to transport the nucleus replacement material.

McAfee does not teach flowing a fluid nucleus pulposus replacement material through the pedicle channels to the disc space.

U.S. Pat. No. 6,921,403 (“Cragg”) discloses accessing the nucleus pulposus by an axial approach from the sacrum. See FIG. 13. Cragg does not disclose accessing the nucleus via the pedicle, nor repairing the endplate after access has been made.

### SUMMARY OF THE INVENTION

The present invention provides a method of filling the disc with flowable nucleus pulposus augmentation material without damaging the annulus fibrosus. In particular, the present invention creates a throughbore through an adjacent vertebral body that accesses the disc through the vertebral endplate. This throughbore is then used as a conduit to fill the disc with a flowable augmentation material. After the disc is filled, the damage to the breached vertebral endplate is repaired. However, because the endplate may be repaired with well known techniques for repairing bone, there is much less risk of extravasation or leakage when compared to that associated with the repair of a damaged annulus fibrosus.

Therefore, in accordance with the present invention, there is provided a method of augmenting a nucleus pulposus in an intervertebral disc between first and second vertebrae, comprising the steps of:
a) creating a throughbore from a pedicle of the first vertebra through an endplate of the first vertebra to access the nucleus pulposus, and
b) filling the disc with a flowable augmentation material through the throughbore.

Also in accordance with the present invention, there is provided a method of augmenting a nucleus pulposus in an intervertebral disc between first and second vertebrae, comprising the steps of:
a) creating a throughbore through an endplate of the first vertebra to access the nucleus pulposus, and
b) repairing the endplate of the first vertebra.

### DESCRIPTION OF THE FIGURES

FIG. 1 discloses a shape memory tube having an obdurator within its bore, wherein the shape memory tube is inserted into a vertebral body via the pedicle.

FIG. 2 discloses the shape memory tube of FIG. 1 having the obdurator removed from its bore.

FIG. 3 discloses the shape memory tube of FIG. 2 now curved towards an endplate.

FIG. 4 discloses the shape memory tube of FIG. 3 having a flexible drill within its bore.

FIG. 5 discloses the shape memory tube of FIG. 3 having a vacuum tube within its bore.

### DETAILED DESCRIPTION OF THE INVENTION

The present invention may be used for all intervertebral discs that require disc augmentation, including herniated discs and degenerated discs. This method is especially useful in discs that do not possess an established weakness in the annulus fibrosus (such as a weakness associated with herniation or a breach), as the method of the present invention preserves the integrity of those intact discs.

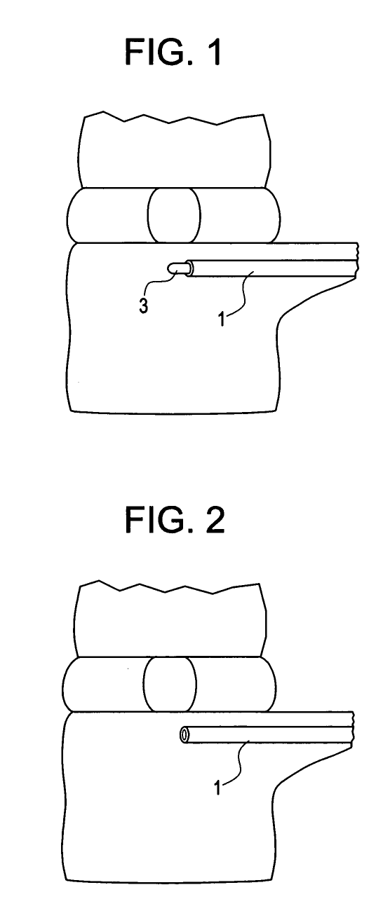

Now referring to FIG. 1, in preferred embodiments, a tube 1 comprising a shape memory material is used to create the throughbore from a pedicle of the first vertebra through an endplate of the first vertebra. Essentially, the tube enters the vertebral body is a substantially straight form. Once in place, the distal end of the shape memory tube curves towards one of the vertebral endplates. This curved tube thereby creates a passage for the flexible drill and a conduit for both disc tissue removal and augmentation material filling. The shape memory material can be either a metal (such as nitinol) or a polymer.

In some embodiments thereof, the memory metal has a martinsitic M→austentic A phase change between 22° C. and 37° C. In some embodiments, the tube is designed so that it is substantially straight at room temperature (˜22° C.) and curved at body temperature (˜37° C.). When the memory metal has such a characteristic, the tube can be made so that its martinsitic state describes a straight shape and its austentic state describes a curved shape. Therefore, the tube can be delivered to the vertebral body in a straight, martinsitic state and in minimally invasive fashion and then undergo austenitic change to a curved state upon body heating so that the tube creates a curved throughbore within the vertebral body so that the distal end of the tube is directed to the vertebral endplate.

In some embodiments thereof, the memory metal has a superelastic property between the temperatures of 22° C. and 37° C. The superelastic property allows the tube to withstand high stresses without experiencing plastic deformation or rupture. When the memory metal has such a superelastic characteristic, a curved tube can be deformed into a straight shape and held in that shape by a obdurator without deformation or rupture. As the obdurator is within and the tube is freed, the tube regains its originally curved shape so that the distal end of the tube is directed to the endplate of the vertebral body.

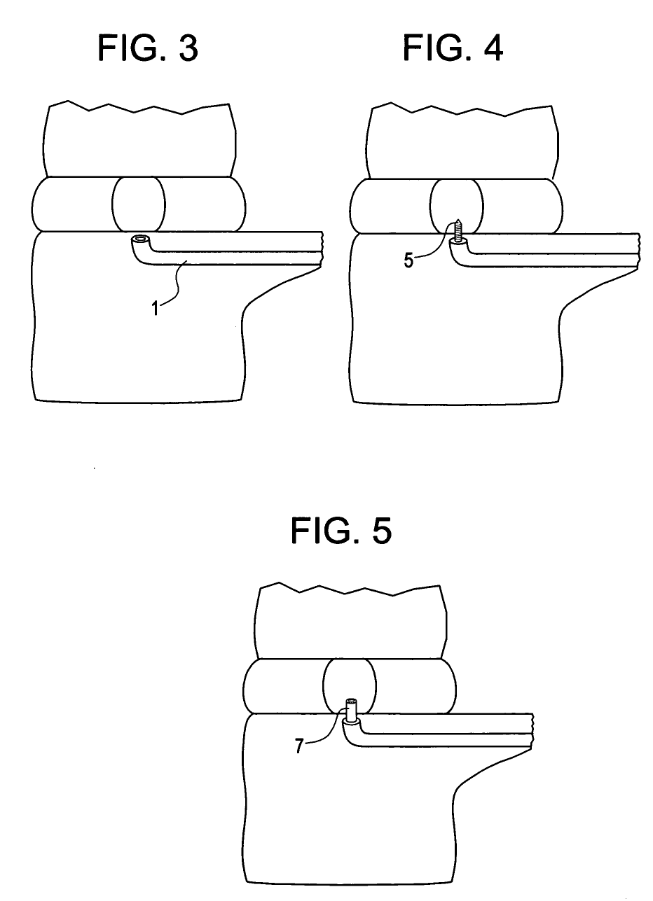

The obdurator of the present invention is used to provide easy entry of the shape memory tube into the vertebral body. The obdurator 3 fits within the shape memory tube and insures that the tube is straight during entry. The assembly is advanced into the vertebral body through a pedicle. Now referring to FIG. 2, once the memory tube is sufficiently advanced, the obdurator is removed and, now referring to FIG. 3, the distal end of the shape memory tube is allowed to curve towards the vertebral endplate.

Now referring to FIG. 4, once the shape memory tube is put in place and is properly oriented towards an endplate, a flexible drill 5 is inserted into the tube and advanced towards the target endplate. Upon contact with the target endplate, the drill is activated and removes bone from the endplate to create access to the disc.

Now referring to FIG. 5, once the drill has removed the hard tissue from the vertebral endplate and opened access to the disc, a tube having vacuum means is advanced through the throughbore and into the disc. Upon entry into the disc (preferably the nucleus pulposus), the vacuum means is activated and nucleus pulposus tissue is removed from the disc to create a cavity within the disc.

Next, a fill tube having an injection port is advanced through the throughbore and into the disc. Upon entry into the disc cavity just created, the augmentation material is injected through the fill tube and into the cavity to at least partially fill the disc.

In some embodiments, the tube associated with the vacuum means is used as the fill tube. In some embodiment, the shape memory tube is used as the fill tube.

The present invention is also particularly useful in patients that are receiving posterior instrumentation in addition to disc augmentation. In one preferred embodiment, the pedicular screw holes used to fasten the posterior instrumentation such as a pedicle screw to the spine can also be used as the vertebral throughbore used to fill the disc. Thus, in some embodiments, the outer diameter of the fill tube is less than the outer diameter of the pedicle screw.

In some embodiments, the posterior instrumentation is posterior dynamic stabilization, preferably comprising two pedicle screws.

Therefore, in accordance with the present invention, there is provided a kit comprising:
a) a flowable nucleus pulposus augmentation material,
b) a fill tube, and
c) an apparatus comprising a pedicle screw.

In some embodiments, access to the nucleus pulposus is accomplished by accessing an adjacent disc (for the purpose of fusing that level or providing a motion disc at that level) and through boring completely through the upper and lower endplates of the intermediate vertebral body.

Therefore, in accordance with the present invention, there is provided a method of augmenting a first nucleus pulposus in an intervertebral disc between first and second vertebrae, comprising the steps of:
a) accessing a first nucleus pulposus between the first and second vertebrae,
b) removing at least a portion of the first nucleus pulposus to create a first disc space,
c) creating a throughbore from a first endplate of the first vertebra through the second endplate of the first vertebra to access a second nucleus pulposus,
d) removing at least a portion of the second nucleus pulposus to create a second disc space,
e) filling the first disc space with a component selected from the group consisting of a flowable augmentation material, a motion disc and a fusion device, and
f) filling the second disc space with a component selected from the group consisting of a flowable augmentation material, a motion disc and a fusion device.

In some embodiments, the flowable augmentation material is non-resorbable. These materials are designed to remain in place for the lifetime of the patient. Suitable non-resorbable augmentation materials include silicone-based materials, polyurethane, polyethylene terephthalate, polycarbonate, thermoplastic elastomers and copolymers such as ether-ketone polymers such as poly(etheretherketone).

Hydrogels useful in the practice of the invention include lightly cross-linked biocompatible homopolymers and copolymers of hydrophilic monomers such as 2-hydroxyalkyl acrylates and methacrylates, e.g., 2-hydroxyethyl methacrylate (HEMA); N-vinyl monomers, for example, N-vinyl-2-pyrrolidone (N-VP); ethylenically unsaturated acids, for example, methacrylic acid (MA) and ethylenically unsaturated bases such as 2-(diethylamino)ethyl methacrylate (DEAEMA). The copolymers may further include residues from non-hydrophilic monomers such as alkyl methacrylates, for example, methyl methacrylate (MMA), and the like. The cross-linked polymers are formed, by known methods, in the presence of cross-linking agents, such as ethyleneglycol dimethacrylate and methylenebis(acrylamide), and initiators such as 2,2-azobis(isobutyronitrile, benzoyl peroxide, and the like, and radiation such as UV and gamma-ray.

In some embodiments, the throughbore of the present invention is used to a deliver bone cement into the damaged endplate. The bone cement may be any material typically used to augment vertebral bodies, including acrylic-based bone cements (such as PMMA-based bone cements), pastes comprising bone particles (either mineralized or demineralized or both; and ceramic-based bone cements (such as HA and TCP-based pastes). In some embodiments, the bone cement comprises the bone cement disclosed in WO 02/064062 (Voellmicke).

In some embodiments, the damage to the vertebral endplate is repaired by applying a bone growth agent to the damaged region.

For the purposes of the present invention, the terms “bone-forming agent” and “bone growth agent” are used interchangeably. Typically, the bone-forming agent may be:
a) a growth factor (such as an osteoinductive or angiogenic factor),
b) osteoconductive (such as a porous matrix of granules),
c) osteogenic (such as viable osteoprogenitor cells), or
d) plasmid DNA.

In some embodiments, the formulation comprises a liquid carrier, and the bone forming agent is soluble in the carrier.

In some embodiments, the bone forming agent is a growth factor. As used herein, the term “growth factor” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4; members of the platelet-derived growth factor (PDGF) family, including PDGF-AB, PDGF-BB and PDGF-AA; EGFs; VEGF; members of the insulin-like growth factor (IGF) family, including IGF-I and -II; the TGF-β superfamily, including TGF-β1, 2 and 3; osteoid-inducing factor (OIF), angiogenin(s); endothelins; hepatocyte growth factor and keratinocyte growth factor; members of the bone morphogenetic proteins (BMPs) BMP-1, BMP-3, BMP-2, OP-1, BMP-2A, BMP-2B, BMP-7 and BMP-14, including MP-52; HBGF-1 and HBGF-2; growth differentiation factors (GDFs), including GDF-5, members of the hedgehog family of proteins, including indian, sonic and desert hedgehog; ADMP-1; bone-forming members of the interleukin (IL) family; GDF-5; and members of the colony-stimulating factor (CSF) family, including CSF-1, G-CSF, and GM-CSF; and isoforms thereof.

In some embodiments, the growth factor is selected from the group consisting of TGF-β, bFGF, and IGF-1. These growth factors are believed to promote the regeneration of bone. In some embodiments, the growth factor is TGF-β. More preferably, TGF-β is administered in an amount of between about 10 ng/ml and about 5000 ng/ml, for example, between about 50 ng/ml and about 500 ng/ml, e.g., between about 100 ng/ml and about 300 ng/ml.

In some embodiments, platelet concentrate is provided as the bone forming agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the bone, and because its fibrin matrix provides a suitable scaffold for new tissue growth.

In some embodiments, the bone forming agent comprises an effective amount of a bone morphogenic protein (BMP). BMPs beneficially increasing bone formation by promoting the differentiation of mesenchymal stem cells (MSCs) into osteoblasts and their proliferation.

In some embodiments, between about 1 ng and about 10 mg of BMP are intraosseously administered into the target bone. In some embodiments, between about 1 microgram (μg) and about 1 mg of BMP are intraosseously administered into the target bone.

In some embodiments, the bone forming agent comprises an effective amount of a fibroblast growth factor (FGF). FGF is a potent mitogen and is angiogenic, and so attracts mesenchymal stem cells to the target area. It is further believed that FGF stimulates osteoblasts to differentiate into osteocytes.

In some embodiments, the FGF is acidic FGF (aFGF).

In some embodiments, the FGF is basic FGF (bFGF).

In some embodiments, between about 1 microgram (μg) and about 10,000 μg of FGF are intraosseously administered into the target bone. In some embodiments, between about 10 μg and about 1,000 μg of FGF are intraosseously administered into the target bone. In some embodiments, between about 50 μg and about 600 μg of FGF are intraosseously administered into the target bone.

In some embodiments, between about 0.1 and about 4 mg/kg/day of FGF are intraosseously administered into the target bone. In some embodiments, between about 1 and about 2 mg/kg/day of FGF are intraosseously administered into the target bone.

In some embodiments, FGF is intraosseously administered into the target bone in a concentration of between about 0.1 mg/ml and about 100 mg/ml. In some embodiments, FGF is intraosseously administered into the target bone in a concentration of between about 0.5 mg/ml and about 30 mg/ml. In some embodiments, FGF is intraosseously administered into the target bone in a concentration of between about 1 mg/ml and about 10 mg/ml.

In some embodiments, FGF is intraosseously administered into the target bone in an amount to provide a local tissue concentration of between about 0.1 mg/kg and about 10 mg/kg.

In some embodiments, the formulation comprises a hyaluronic acid carrier and bFGF. In some embodiments, formulations described in U.S. Pat. No. 5,942,499 (“Orquest”) are selected as FGF-containing formulations.

In some embodiments, the bone forming agent comprises an effective amount of insulin-like growth factor. IGFs beneficially increase bone formation by promoting mitogenic activity and/or cell proliferation.

In some embodiments, the bone forming agent comprises an effective amount of parathyroid hormone (PTH). Without wishing to be tied to a theory, it is believed that PTH beneficially increases bone formation by mediating the proliferation of osteoblasts.

In some embodiments, the PTH is a fragment or variant, such as those taught in U.S. Pat. No. 5,510,370 (Hock) and U.S. Pat. No. 6,590,081 (Zhang), and published patent application 2002/0107200 (Chang), the entire contents of which are incorporated herein in their entirety. In one embodiment, the PTH is PTH (1-34) (teriparatide), e.g., FORTEO® (Eli Lilly and Company). In some embodiments, the BFA is a parathyroid hormone derivative, such as a parathyroid hormone mutein. Examples of parathyroid muteins are discussed in U.S. Pat. No. 5,856,138 (Fukuda), the entire contents of which are incorporated herein in its entirety.

In some embodiments, the bone forming agent comprises an effective amount of a statin. Without wishing to be tied to a theory, it is believed that statins beneficially increase bone formation by enhancing the expression of BMPs.

In some embodiments, the bone forming agent is a porous matrix, and is preferably injectable. In some embodiments, the porous matrix is a mineral. In one embodiment, this mineral comprises calcium and phosphorus. In some embodiments, the mineral is selected from the group consisting of calcium phosphate, tricalcium phosphate and hydroxyapatite. In one embodiment, the average porosity of the matrix is between about 20 and about 500 μm, for example, between about 50 and about 250 μm. In yet other embodiments of the present invention, in situ porosity is produced in the injected matrix to produce a porous scaffold in the injected fracture stabilizing cement. Once the in situ porosity is produced in the target tissue, the surgeon can inject other therapeutic compounds into the porosity, thereby treating the surrounding tissues and enhancing the remodeling process of the target tissue and the injectable cement.

In some embodiments, the mineral is administered in a granule form. It is believed that the administration of granular minerals promotes the formation of the bone growth around the minerals such that osteointegration occurs.

In some embodiments, the mineral is administered in a settable-paste form. In this condition, the paste sets up in vivo, and thereby immediately imparts post-treatment mechanical support to the fragile OP body.

In another embodiment, the treatment is delivered via injectable absorbable or non-absorbable cement to the target tissue. The treatment is formulated using bioabsorbable macro-sphere technologies, such that it will allow the release of the bone forming agent first, followed by the release of the anti-resorptive agent. The cement will provide the initial stability required to treat pain in fractured target tissues. These tissues include, but are not limited to, hips, knee, vertebral body fractures and iliac crest fractures. In some embodiments, the cement is selected from the group consisting of calcium phosphate, tricalcium phosphate and hydroxyapatite. In other embodiments, the cement is any hard biocompatible cement, including PMMA, processed autogenous and allograft bone. Hydroxylapatite is a preferred cement because of its strength and biological profile. Tricalcium phosphate may also be used alone or in combination with hydroxylapatite, particularly if some degree of resorption is desired in the cement.

In some embodiments, the porous matrix comprises a resorbable polymeric material.

In some embodiments, the bone forming agent comprises an injectable precursor fluid that produces the in situ formation of a mineralized collagen composite. In some embodiments, the injectable precursor fluid comprises:
a) a first formulation comprising an acid-soluble type I collagen solution (preferably between about 1 mg/ml and about 7 mg/ml collagen) and
b) a second formulation comprising liposomes containing calcium and phosphate.

Combining the acid-soluble collagen solution with the calcium- and phosphate-loaded liposomes results in a liposome/collagen precursor fluid, which, when heated from room temperature to 37° C., forms a mineralized collagen gel.

In some embodiments, the liposomes are loaded with dipalmitoylphosphatidylcholine (90 mol %) and dimyristoyl phosphatidylcholine (10 mol %). These liposomes are stable at room temperature but form calcium phosphate mineral when heated above 35° C., a consequence of the release of entrapped salts at the lipid chain melting transition. One such technology is disclosed in Pederson, Biomaterials 24: 4881-4890 (2003), the specification of which is incorporated herein by reference in its entirety.

Alternatively, the in situ mineralization of collagen could be achieved by an increase in temperature achieved by other types of reactions including, but not limited to, chemical, enzymatic, magnetic, electric, photo- or nuclear. Suitable sources thereof include light, chemical reaction, enzymatically controlled reaction and an electric wire embedded in the material. To further elucidate the electric wire approach, a wire (which can be the reinforcement rod) can first be embedded in the space, heated to create the calcium deposition, and then withdrawn. In some embodiments, this wire may be a shape memory such as nitinol that can form the shape. Alternatively, an electrically-conducting polymer can be selected as the temperature raising element. This polymer is heated to form the collagen, and is then subject to disintegration and resorption in situ, thereby providing space adjacent the mineralized collagen for the bone to form.

In one embodiment, the bone forming agent is a plurality of viable osteoprogenitor cells. Such viable cells, introduced into the bone, have the capability of at least partially repairing any bone loss experienced by the bone during the osteoporotic process. In some embodiments, these cells are introduced into the cancellous portion of the bone and ultimately produce new cancellous bone. In others, these cells are introduced into the cortical region and produce new cortical bone.

In some embodiments, these cells are obtained from another human individual (allograft), while in other embodiments, the cells are obtained from the same individual (autograft). In some embodiments, the cells are taken from bone tissue, while in others, the cells are taken from a non-bone tissue (and may, for example, be mesenchymal stem cells, chondrocytes or fibroblasts). In others, autograft osteocytes (such as from the knee, hip, shoulder, finger or ear) may be used.

In one embodiment, when viable cells are selected as an additional therapeutic agent or substance, the viable cells comprise mesenchymal stem cells (MSCs). MSCs provide a special advantage for administration into an uncoupled resorbing bone because it is believed that they can more readily survive the relatively harsh environment present in the uncoupled resorbing bone; that they have a desirable level of plasticity; and that they have the ability to proliferate and differentiate into the desired cells.

In some embodiments, the mesenchymal stem cells are obtained from bone marrow, such as autologous bone marrow. In others, the mesenchymal stem cells are obtained from adipose tissue, preferably autologous adipose tissue.

In some embodiments, the mesenchymal stem cells injected into the bone are provided in an unconcentrated form, e.g., from fresh bone marrow. In others, they are provided in a concentrated form. When provided in concentrated form, they can be uncultured. Uncultured, concentrated MSCs can be readily obtained by centrifugation, filtration, or immuno-absorption. When filtration is selected, the methods disclosed in U.S. Pat. No. 6,049,026 (“Muschler”), the specification of which is incorporated herein by reference in its entirety, can be used. In some embodiments, the matrix used to filter and concentrate the MSCs is also administered into the uncoupled resorbing bone.

In some embodiments, bone cells (which may be from either an allogeneic or an autologous source) or mesenchymal stem cells, may be genetically modified to produce an osteoinductive bone anabolic agent which could be chosen from the list of growth factors named herein. The production of these osteopromotive agents may lead to bone growth.

In some embodiments, the osteoconductive material comprises calcium and phosphorus. In some embodiments, the osteoconductive material comprises hydroxyapatite. In some embodiments, the osteoconductive material comprises collagen. In some embodiments, the osteoconductive material is in a particulate form.

Recent work has shown that plasmid DNA will not elicit an inflammatory response as does the use of viral vectors. Genes encoding bone (anabolic) agents such as BMP may be efficacious if injected into the uncoupled resorbing bone. In addition, overexpression of any of the growth factors provided herein or other agents which would limit local osteoclast activity would have positive effects on bone growth. In one embodiment, the plasmid contains the genetic code for human TGF-β or erythropoietin (EPO).

Accordingly, in some embodiments, the additional therapeutic agent is selected from the group consisting of viable cells and plasmid DNA.

### EXAMPLE I

A patient with discogenic back pain with a black disc at L 4-5 is to be treated with disc augmentation along with a motion preserving posterior instrumentation system. The patient is prepped for a posterior approach. The pedicles are tapped at L4 and L5 to prepare for screw placement. A Nitonol tube with a harp tipped obdurator is inserted through one of the pedicle screw holes at L5 and advanced into the vertebral body. The nitinol tube system curves upwards so as to contact the endplate. The obdurator is then removed and replaced with a flexible drill to create a hole through the endplate into the disc. The drill is then removed and the disc contents are partially or fully removed by applying suction through a tip inserted through the Nitinol tube. The vacuum tip is then removed and the disc augmentation material is then inserted through the nitinol tube into the disc space. The hole in the endplate is then repaired using an in-situ settable calcium phosphate paste. The nitinol tube is then removed and replaced with a pedicle screw.

### EXAMPLE 2

The same procedure is used as in Example 1, except that access to the disc space is also created from one of the L4 pedicle holes. This port is used to introduce a mechanical tool to help in the evacuation of the disc space. Also, this port is used to release pressure, if necessary, during the introduction of the disc augmentation material into the disc space.

### EXAMPLE 3

A procedure substantially similar to that used in Examples 1 and 2 is used, except that posterior instrumentation is used to distract the disc prior to performing the disc evaluation and disc augmentation. The distraction is achieved by using one pedicle screw at each level, leaving the other hole accessible.

### EXAMPLE 4

A procedure substantially similar to that used in Examples 1 and 2 is used, except that cannulated pedicle screws are used.

Therefore, also in accordance with the present invention, there is provided a kit comprising:
a) a flowable nucleus pulposus augmentation material, and
b) a cannulated pedicle screw.

I claim:

1. A method of augmenting a nucleus pulposus in an intervertebral disc between first and second vertebrae, comprising the steps of:
a) creating a throughbore from a pedicle of the first vertebra through an endplate of the first vertebra to access the nucleus pulposus, and
b) filling the disc with a flowable augmentation material through the throughbore.

2. The method of claim 1 further comprising, prior to step b), the step of:
c) removing at least a portion of the nucleus pulposus through the throughbore to form a space in the intervertebral disc.

3. The method of claim 1 further comprising the step of:
c) inserting a pedicle screw into the throughbore.

4. The method of claim 1 wherein the step of creating a throughbore includes inserting a shape memory tube into the pedicle.

5. The method of claim 4 wherein an obdurator is provided within the shape memory tube during the insertion step.

6. The method of claim 1 wherein the step of creating a throughbore includes inserting a flexible drill through the shape memory tube.

7. The method of claim 6 further comprising, prior to step b), the step of:
c) removing at least a portion of the nucleus pulposus through the throughbore to form a space in the intervertebral disc.

8. The method of claim 7 wherein the step of removing at least a portion of the nucleus pulposus includes applying a vacuum to nucleus pulposus through the throughbore.

9. The method of claim 1 wherein the step of filling the disc includes advancing a fill tube having an injection port through the throughbore.

10. A method of augmenting a nucleus pulposus in an intervertebral disc between first and second vertebrae, comprising the steps of:
a) creating a throughbore through an endplate of the first vertebra to access the nucleus pulposus, and
b) repairing the endplate of the first vertebra.

11. The method of claim 10 wherein the endplate is repaired with a bone cement.

12. The method of claim 11 wherein the bone cement is selected from the group consisting of acrylic-based bone cements, pastes comprising bone particles; and ceramic-based bone cements.

13. The method of claim 12 wherein the bone cement is an acrylic-based bone cement.

14. The method of claim 12 wherein the bone cement is a paste comprising bone particles.

15. The method of claim 12 wherein the bone cement is a ceramic-based bone cement.

16. The method of claim 10 wherein the endplate is repaired with a bone growth agent.

17. The method of claim 16 wherein the bone growth agent is a resorbable scaffold.

18. The method of claim 16 wherein the bone growth agent is a growth factor.

19. The method of claim 16 wherein the bone growth agent are cells.

20. The method of claim 10 wherein the throughbore is created from a pedicle of the first vertebra through an endplate of the first vertebra.

21. A kit comprising:
a) a flowable nucleus pulposus augmentation material,
b) a fill tube, and
c) an apparatus comprising a pedicle screw.

22. The kit of claim 21 wherein the apparatus is a posterior dynamic stabilization system.

23. The kit of claim 21 wherein the posterior dynamic stabilization system comprises at least two pedicle screws.

24. The kit of claim 21 wherein the fill tube has an outer diameter and the pedicle screw has an outer diameter, wherein the outer diameter of the fill tube is less than the outer diameter of the pedicle screw.

25. The kit of claim 21 wherein the augmentation material is selected from the group consisting of a silicone-based material, polyurethane, polyethylene terephthalate, polycarbonate, a thermoplastic elastomer, a hydrogel and a copolymer.

26. A kit comprising:
a) a flowable nucleus pulposus augmentation material, and
b) a cannulated pedicle screw.

27. A method of augmenting a first nucleus pulposus in an intervertebral disc between first and second vertebrae, comprising the steps of:
a) accessing a first nucleus pulposus between the first and second vertebrae,
b) removing at least a portion of the first nucleus pulposus to create a first disc space,
c) creating a throughbore from a first endplate of the first vertebra through the second endplate of the first vertebra to access a second nucleus pulposus,
d) removing at least a portion of the second nucleus pulposus to create a second disc space,
e) filling the first disc space with a component selected from the group consisting of a flowable augmentation material, a motion disc and a fusion device, and
f) filling the second disc space with a component selected from the group consisting of a flowable augmentation material, a motion disc and a fusion device.
